(19) United States
(12) Patent Application Publication
(10) Pub. No.: US 2010/0120907 A1
(43) Pub. Date: May 13, 2010

(54) DERIVATIVES OF AMYRIS ALCOHOLS AND EUDESMOL FOR TREATING COLD SORES AND HERPES

(76) Inventors: Chandra Ulagaraj Singh, San Antonio, TX (US); Rao Jagaveerabhadra Nulu, Austin, TX (US)

Correspondence Address: REED SMITH LLP, 2500 ONE LIBERTY PLACE, 1650 MARKET STREET, PHILADELPHIA, PA, 19103, US

(21) Appl. No.: 12/450,902

(22) PCT Filed: Apr. 18, 2008
(86) PCT No.: PCT/US08/60878

§ 371 Date: Jan. 11, 2010

Related U.S. Application Data
(60) Provisional application No. 60/912,883, filed on Apr. 19, 2007.

(51) Int. Cl. A61K 31/215; A61K 31/22; A61P 35/00; A61P 17/00; C07C 69/74; C07C 69/52; C07C 69/00

(52) U.S. Cl. 514/510; 514/530; 514/531; 514/529; 514/549; 514/552; 514/546; 560/122; 560/123; 560/124; 560/220; 560/129

(57) ABSTRACT

Provided are topical formulations comprising an Amyris alcohol and/or ester derivatives of Amyris alcohol which may be used for the treatment of diseases including herpes virus infection (e.g., HSV-1, HSV-2), epidermoid carcinoma, cold sores, and human papillomavirus. Amyris alcohols contemplated for use with the present invention include valerianol, beta-eudesmol, epi-gamma-eudesmol, elemol, alpha-eudesmol, and ester derivatives thereof.

This application claims priority to U.S. Application No. 60/912,883 filed on Apr. 19, 2007, the entire disclosure of which is specifically incorporated herein by reference in its entirety without disclaimer.

### BACKGROUND OF THE INVENTION

1. Field of the Invention

The present invention relates generally to the fields of pharmacology and medicine. More particularly, it concerns ester derivatives of amyris alcohols for the treatment of diseases including herpes simplex infection.

2. Description of Related Art

Oral herpes, an infection caused by the herpes simplex virus, is estimated to be present in 50 to 80 percent of the American adult population. Nearly 20 percent, over 50 million people, are infected with genital herpes, also caused by the herpes simplex virus, and the majority of these cases may be unaware they even have it. Studies show that more than 500,000 Americans are diagnosed with genital herpes each year, and the largest increase is occurring in young teens.

Cold sores, sometimes called fever blisters, are clusters of small blisters on the lip and outer edge of the mouth. The skin around the blisters is often red and inflamed. The blisters can break open, weep a clear fluid, and then scab over after a few days. Complete healing may take 7 to 10 days. Cold sores are caused by the herpes simplex virus (HSV). There are two types of herpes simplex virus (HSV). HSV-1 usually leads to lip and mouth sores (herpes labialis), while HSV-2 most often leads to genital herpes. However, both virus types can cause cold sores or genital herpes if skin comes into contact with either type. Cold sores are caused by the herpes simplex virus (HSV). There are two types of herpes simplex virus (HSV). HSV-1 usually leads to lip and mouth sores (herpes labialis), while HSV-2 most often leads to genital herpes. However, both virus types can cause cold sores or genital herpes if skin comes into contact with either type. Cold sores are estimated to be present in 50 to 80 percent of the American adult population. Nearly 20 percent, over 50 million people, are infected with genital herpes, also caused by the herpes simplex virus, and the majority of these cases may be unaware they even have it. Studies show that more than 500,000 Americans are diagnosed with genital herpes each year, and the largest increase is occurring in young teens.

There is no cure for cold sores and genital herpes to date. Efforts to develop a herpes vaccine by biotechnology companies are ongoing. Until an effective herpes vaccine or cure for HSV infection is found, the prevailing approach to treatment continues to be suppressive antiviral therapy.

Topical creams are commonly used to treat cold sores. Many are prescription medications that can slightly shorten the duration of cold sores, usually by just 1 to 2 days. Studies are ongoing to determine the effectiveness of these creams (Boon et al., 2000). Some experts find that even when nonprescription topical creams are used frequently, every 2 hours during wake time, at the first sign of an outbreak, they may only speed recovery time by a few hours or a day (Habif et al., 2004).

Penciclovir cream (such as Denavir) is an antiviral cream that may reduce healing time by 1 to 2 days, especially if the cold sore was triggered by sunlight exposure (Sacks et al., 2001; Herpes, 2003). It also reduces pain, itching, burning, and tenderness associated with cold sores. Penciclovir cream may cause side effects such as mild pain or stinging when it is applied. It is possible, although rare, that the cream may also cause a skin rash or headache.

Acyclovir ointment and cream are used up to six times a day for 10 days. Treatment with acyclovir ointment works best if it is used at the first sign of cold sore symptoms. The cream can improve healing time by up to ½ a day. Side effects of acyclovir ointment may include mild pain or stinging at the site of application. The cream may cause temporary skin irritation.

Tetracaine cream is a nonprescription topical anesthetic that lessens the physical sensation and can relieve pain and itching associated with cold sores. Initial studies show that tetracaine cream can reduce the healing time of cold sores by up to 2 days (Habif et al., 2001). Tetracaine cream is applied to cold sores up to six times daily for best results. Pain and itching are relieved usually within 2 to 3 days after first applying tetracaine cream.

Docosanol 10% (Abreva) is a newer nonprescription cream that is safe and effective for treating cold sores. It is most effective when applied at the first signs of a cold sore outbreak (Sacks et al., 2001). It is the first nonprescription cold sore medication approved by the U.S. Food and Drug Administration (FDA) to shorten healing time and duration of symptoms.

Valacyclovir was recently approved by the U.S. Food and Drug Administration (FDA) as a one-day treatment to reduce cold sore duration in people 12 years and older (Habif et al., 2004). It is absorbed by the body much better than some other antiviral medications (such as acyclovir). Possible side effects include skin rash, allergic reaction, headache, dizziness, insomnia, depression, and fatigue.

Despite the foregoing, cold sores and herpes virus infection continue to present a significant problem. Clearly, there exists a need for new therapies to treat cold sores and herpes virus infection.

### SUMMARY OF THE INVENTION

The present invention overcomes limitations in the prior art by providing new treatments for diseases including cold sores and herpes virus infection. In particular, it has been discovered that the amyris alcohols and esterified amyris alcohol derivatives are particularly effective in treating cold sores and genital herpes in humans.

The amyris alcohol ester derivatives may have certain advantages for the treatment of diseases such as treating cold sores and/or herpes virus symptoms. For example, esterified amyris alcohol derivatives are in certain embodiments highly lipophilic, and, without wishing to be bound by any theory, it is envisioned that these compounds may be enzymatically cleaved after administration to a mammal or human patient to form the corresponding amyris alcohol in vivo. In particular, esterified amyris alcohols may have high lipophilicity, be non-irritating to the skin, have improved stability, and have improved bioavailability when administered topically as compared to amyris oil. Amyris alcohols may be stable and relatively non-toxic. As shown in the below examples, it has been discovered that amyris alcohol and ester derivatives such as amyris acetate and amyris propanoate have therapeutic utility in treating cold sores and genital herpes in humans.

The present invention generally pertains to amyris alcohol or a compound of formula (I):

R—CO—O₁Am (I)

wherein O₁Am refers to an oxygen present in an alcohol group of the corresponding amyris alcohol. In other words, the invention pertains to esters of amyris alcohols. These compounds may be used to treat a disease in a mammal. If an amyris alcohol is administered, then the mammal may be a human and the disease may be selected from the group consisting of cold sores, genital herpes, herpes simplex virus infection, HSV-1 infection, HSV-2 infection, epidermoid carcinoma, and human papillomavirus (HPV) tumors.

In formula I, R may be selected from the group consisting of C₁-C₁₈ alkyl, C₁-C₁₈ aryl, C₁-C₁₈ alkylene (e.g., an alkenyl or an alkynyl), C₁-C₁₈ substituted alkyl, C₁-C₁₈ substituted aryl, and C₁-C₁₈ substituted alkylene. The alkyl, aryl and alkylene groups may be substituted or unsubstituted, branched or straight chains. In addition, R may contain heteroatoms and may be straight chained or branched.

Examples of suitable straight-chain alkyl groups in formula I include methyl, ethyl, propyl, butyl, hexyl, heptyl, octyl, dodecyl, 1-pentadecyl, 1-heptadecyl and the like groups. Examples of suitable branched chain alkyl groups in formula I include isopropyl, sec-butyl, t-butyl, 2-methylbutyl, 2-pentyl, 3-pentyl and the like groups. Examples of suitable cyclic alkyl groups in formula I include cyclopropyl, cyclobutyl, cyclopentyl and cyclohexyl groups. Examples of suitable “alkenyl” groups in formula I include vinyl(ethenyl), 1-propenyl, i-butenyl, pentenyl, hexenyl, n-decenyl and c-pentenyl and the like. The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

By the expression “phenalkyl groups wherein the alkyl moiety contains 1 to 3 or more carbon atoms” is meant benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” in formula I are pyridinyl, thienyl or imidazolyl. As noted herein, the expression “halo” refers to halogens, which include F, Cl, Br, and I. Among the compounds represented by the general formula I, preferred compounds are such in which R is one of the following groups: methyl, ethyl, propyl, butyl, pentyl, hexyl, 1-pentadecyl, 1-heptadecyl, isobutyl, methoxyethyl, ethoxyethyl, benzyl and nicotinyl.

The compounds of formula I are esters of alcohols present in amyris alcohol. To the knowledge of the inventors, the literature does not disclose or indicate that esters of amyris alcohols have utility as pro-drug forms suitable for oral and topical delivery for treating diseases such as cold sores and genital herpes.

The amyris alcohol may be administered to the human. In certain embodiments, the amyris alcohol is selected from the group consisting of valerianol, beta-eudesmol, epi-gamma-eudesmol, elemol, or alpha-eudesmol.

In certain embodiments, the esterified amyris alcohol is administered to the mammal. In various embodiments, the esterified amyris alcohol is be selected from the group consisting of:

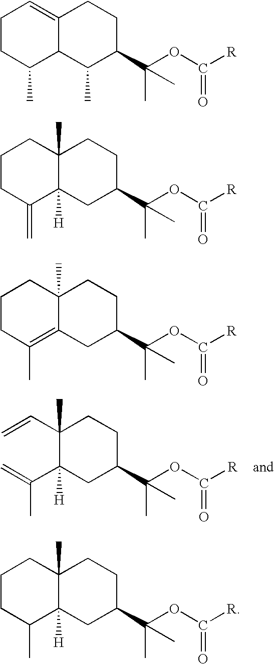

The disease may be selected from the group consisting of cold sores, genital herpes, herpes simplex virus infection, HSV-1 infection, HSV-2 infection, epidermoid carcinoma, and human papillomavirus (HPV) tumors. For example, the disease may be a tumor induced by a human papillomavirus (HPV) selected from the group consisting of verrucae warts, plantar warts, flat warts, genital warts and Molluscum contagiosum.

Another aspect of the present invention relates to pharmaceutical compositions comprising, e.g., an amyris alcohol or esterified amyris alcohol. The pharmaceutical composition may be comprised in a topical formulation such as, e.g., a cream, lotion, spray, wipe, or drop formulation. The pharmaceutical composition may comprise one or more additional pharmaceutical agents. The additional pharmaceutical agent may be a fungicidal or fungistatic agent, a bacteriocidal or bacteriostatic agent, a viricidal or virostatic agent, or a cytotoxic agent. The pharmaceutical composition may further comprise one or more pharmaceutically acceptable excipients. The excipients may include one or more pharmaceutically acceptable antioxidants such as, e.g., ascorbic acid, sodium ascorbate, sodium bisulfate, sodium metabisulfate, curcumin, curcumin derivatives, ursolic acid, resveratrol, resveratrol derivatives, alpha-lipoic acid or monothioglycerol. The excipients may include one or more pharmaceutically acceptable preservatives and/or buffering agents. The buffering agent may be monobasic and dibasic sodium phosphate, sodium benzoate, potassium benzoate, sodium citrate, sodium acetate or sodium tartrate. The preservative may be methylparaben, methylparaben sodium, propylparaben, propylparaben sodium, benzalkonium chloride or benzthonium chloride. The composition may comprise one or more pharmaceutically acceptable polysaccharides such as, e.g., dextran sulfate, pectin, modified pectin, insoluble 1,3-β-D glucan, micronized 1,3-β-D glucan, soluble 1,3-β-D glucan, phosphorylated 1,3-β-D glucan, aminated 1,3-β-D glucan and carboxymethylated 1,3-β-D glucan, sulfated 1,3-β-D glucan, insoluble 1,3/1,6-β-D glucan, micronized 1,3/1,6-β-D glucan, soluble 1,3/1,6-β-D glucan, phosphorylated 1,3/1,6-β-D glucan, aminated 1,3/1,6-β-D glucan and carboxymethylated 1,3/1,6-β-D glucan or sulfated 1,3/1,6-β-D glucan. The mammal may be a human.

The pharmaceutical composition may comprise from about 1% to about 90%, from about 5% to about 50%, or from about 10% to about 30% by weight of amyris alcohol or an ester of amyris alcohol. The composition may be administered orally, nasally, topically, rectally or vaginally. The pharmaceutical preparation may be sterile or a Good Manufacturing Practice grade (GMP grade) pharmaceutical preparation. The composition may be a cosmetic or topical composition such as, e.g., an emulsion, a cream, a lotion, a solution, an anhydrous composition, a gel, or an ointment.

Accordingly, one aspect of the present invention is to disclose the esters of amyris alcohol in the treatment of cold sores and genital herpes in humans. In certain embodiments, the amyris alcohol is obtained from Amyris balsamifera.

In another aspect of this invention, the esters of an amyris alcohol such as, e.g., eudesmol and/or valerianol, are disclosed for the treatment of cold sores and genital herpes in humans.

Yet another aspect of the present invention is to disclose the esters of amyris alcohol, esters of eudesmol and valerianol in the treatment of warts caused by the human papillomavirus (HPV) in humans.

Yet in another aspect of this invention, the esters of the active component or components of the amyris alcohol, namely eudesmol and valerianol, are disclosed for the treatment of warts caused by the human papillomavirus (HPV) in humans.

In particular, the amyris alcohol or esters of amyris alcohol described herein may be used for the preparation of therapeutic compositions in the treatment of cold sores, genital herpes and warts induced in humans. Preferably, the compositions useful in the method may be topically applied to the human in need of such therapy.

In various embodiments, compositions and methods of the present invention may be used with little or no destruction to healthy, uninfected tissue and little or no local or systemic side effects (e.g., scarring, disfigurement, or discomfort) to the human treated. The amyris alcohol or ester of amyris alcohol may be administered to an area of the human which is anticipated to evidence cold sores or an area which presently exhibits a HSV outbreak (e.g., genital herpes) to alleviate or eliminate the sores.

In accordance with the method according to this invention, regular use of the amyris alcohol or ester of amyris alcohol is meant to mean application of the alcohol or ester at least once a day to the body surface containing the cold sores or genital herpes or viral-induced tumors (i.e., warts and Molluscom contagiosum tumors).

There is further disclosed a method for the prevention and treatment of cold sores and genital herpes, comprising the application of a cream or douche containing amyris alcohol or an ester of amyris alcohol or mixtures thereof, to the affected area of the human body. There is also disclosed a method for treating genital herpes, said method comprising the application of amyris alcohol or an ester of amyris alcohol or mixtures thereof, to the genital area of a human for a period of time and at a sufficient concentration to eradicate the herpes virus from the genital area of the human.

The pharmaceutical compositions of the present invention can additionally include one or more pharmaceutically acceptable excipients. One of ordinary skill in the art would be familiar with pharmaceutically acceptable excipients. For example, the pharmaceutically acceptable excipient may be a water soluble sugar, such as mannitol, sorbitol, fructose, glucose, lactose, and sucrose.

The pharmaceutical compositions of the present invention may further comprise one or more pharmaceutically acceptable antioxidants. Any pharmaceutically acceptable antioxidant known to those of ordinary skill in the art is contemplated for inclusion in the present pharmaceutical compositions. For example, the pharmaceutically acceptable antioxidant may be selected from the group consisting of ascorbic acid, sodium ascorbate, sodium bisulfate, sodium metabisulfate and monothio glycerol.

The pharmaceutical compositions of the present invention may further comprise one or more pharmaceutically acceptable preservatives. Any pharmaceutically acceptable preservative known to those of ordinary skill in the art is contemplated for inclusion in the present pharmaceutical compositions. Examples of such preservatives include methylparaben, methylparaben sodium, propylparaben, propylparaben sodium, benzalkonium chloride, and benzthonium chloride.

The pharmaceutical compositions of the present invention may further comprise one or more pharmaceutically acceptable buffering agents. Any pharmaceutically acceptable buffering agent known to those of ordinary skill in the art is contemplated for inclusion in the present pharmaceutical compositions. Examples of such buffering agents include of monobasic sodium phosphate, dibasic sodium phosphate, sodium benzoate, potassium benzoate, sodium citrate, sodium acetate, and sodium tartrate.

The pharmaceutical compositions of the present invention can include any concentration of a compound of the present invention. For example, the concentration of compound may be 0.1 mg/ml to 1000 mg/ml or greater. In certain particular embodiments, the concentration of compound is 1 mg/ml to 500 mg/ml. In still further embodiments, the concentration of compound is 5 mg/ml to 200 mg/ml.

In some embodiments of the present invention, the pharmaceutical composition includes more than one of the novel compounds set forth above. In other embodiments of the present invention, the pharmaceutical composition includes one or more secondary therapeutic agents directed to a disease or health-related condition.

The present invention also generally pertains to methods of treating or preventing a pathological condition in a subject, comprising providing a therapeutically effective amount of any of the pharmaceutical compositions set forth above, and administering the composition to the subject. The subject can be any subject, such as a mammal or avian species. In certain particular embodiments, the mammal is a human. The human may be an individual affected by or at risk of developing a disease or condition amenable to therapy with amyris alcohol. For example, the pathological condition may be cold sores, genital herpes, genital warts, acne, urinary tract infection, a wound, or skin wrinkling.

The pharmaceutical composition of the present invention may be administered to the subject by any method known to those of ordinary skill in the art. For example, the method of administering the composition to the subject may include oral, topical, nasal, inhalational, rectal, or vaginal. Methods of administration are discussed in greater detail in the specification below.

In certain embodiments of the methods of the present invention, the method involves administering to the subject a therapeutically effective amount of a secondary agent. The secondary agent can be any pharmacologic agent known or suspected to be of benefit in the treatment or prevention of a disease or health-related condition in a subject. For example, in some embodiments, the secondary agent is a antihyperproliferative agent. Antihyperproliferative agents, which include chemotherapeutic agents, are well-known to those of ordinary skill in the art. Examples of such agents which may be used with the present invention include doxorubicin, daunorubicin, mitomycin, actinomycin D, bleomycin, cisplatin, VP16, an enedyine, taxol, vincristine, vinblastine, carmustine, mellphalan, cyclophsophamide, chlorambucil, busulfan, lomustine, 5-fluorouracil, gemcitabin, BCNU, or camptothecin. The secondary agent may be an anti-viral agent. Examples of anti-viral agents include acyclovir, tetracaine, penciclovir, docosanol, and valacyclovir.

It must be noted that, as used in this specification, the singular forms “a,” “an” and “the” include plural referents unless the context clearly dictates otherwise. Thus, for example, reference to “a pharmacologically active agent” includes a combination of two or more pharmacologically active agents, and the like. In describing the present invention, the following terminology will be used in accordance with the definitions set out below.

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned which also induce the desired pharmacologic effect.

The term “topical administration” is used in its conventional sense to mean delivery of a topical drug or pharmacologically active agent to the skin or mucosa.

“Carriers” or “vehicles” as used herein refer to carrier materials suitable for drug administration. Carriers and vehicles useful herein include any such materials known in the art, e.g., any liquid, gel, solvent, liquid diluent, solubilizer, or the like, which is nontoxic and which does not interact with other components of the composition in a deleterious manner.

By an “effective” amount of a drug or pharmacologically active agent is meant a nontoxic but sufficient amount of the drug or agent to provide the desired effect.

The term “amyris alcohol” as used herein refers to the alcohol distilled from the amyris oil, e.g., by vacuum distillation. For example, the volatile organic compounds may be distilled off, leaving the alcohols more concentrated due to their higher boiling point. Thus, amyris alcohol may include a mixture of alcohols present in amyris oil. Alternately individual amyris alcohols or amyris alcohol derivatives may be used with the present invention.

The term “ester of amyris alcohol” refers to the acylated or esterified product of amyris alcohol. The various alcohols present in the amyris alcohol may be fully esterified and thus an “ester of amyris alcohol” may typically contain a mixture of esters.

The term “eudesmol” as used herein is intended to encompass not only α-, β- and γ-eudesmol, but any isomer or any compounded mixture thereof.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: The Chemical Structures of Eudesmol, Valerianol and Elemol.

FIG. 2: Examples of esterified amyris alcohols.

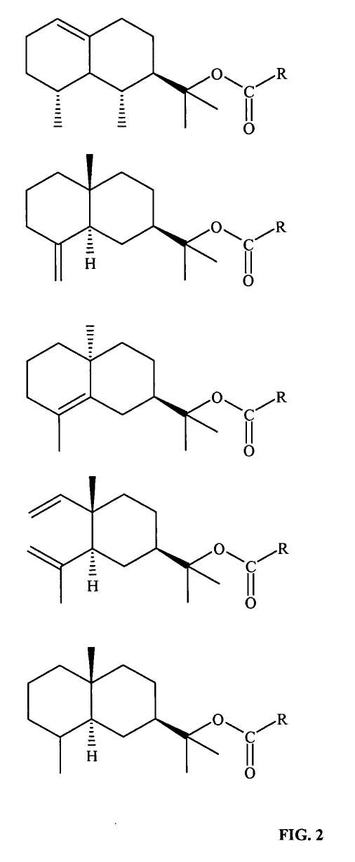

### DESCRIPTION OF ILLUSTRATIVE EMBODIMENTS

The present invention is based on the inventors' discovery that amyris alcohols and/or certain derivatives of amyris alcohols may be used to treat diseases including cold sores, genital herpes, genital warts and urinary tract infection. In various embodiments, the amyris alcohol derivatives used are highly lipophilic, non-irritating, exhibit low toxicity which allow for higher concentrations to be administered, and have improved bioavailability following administration, e.g., in a cream or ointment formulation. Oral and topical formulations are provided which utilize the lipid solubility of the amyris alcohols and amyris alcohol derivatives. As shown in the below examples, these compounds and formulations may be used to treat diseases in human patients.

### I. AMYRIS ALCOHOLS

A member of the Rutaceae family, the amyris tree (Amyris balsamifera) is native to Haiti but is now grown in tropical zones throughout the world. Although Amyris balsamifera is not a member of the santalum (sandalwood) family, it is commercially often referred to as “West Nepal sandalwood” or “West Indian sandalwood.” Amyris iols are thus unrelated to sandalwood oils, and they produce a smell that is distinct from sandalwood oil. Amyris essential oil (Van et al., 1989) commonly referred to as “West Indian Amyris alcohol,” the Botanical origin of the tree yielding this oil remained obscure until 1886. The main country of origin today is Haiti, where the oil is obtained by steam distillation from broken up wood & branches and its chemical composition has been investigated (Van et al., 1991a; 1991b; 1989). Amyris essential oil has been used for wound washes, influenza, child birth recovery, diarrhea, used also as a room fragrance or mood fragrance, as a cheaper alternative to genuine Sandalwood. Used as a fragrance, fixative or a component of soap fragrance.

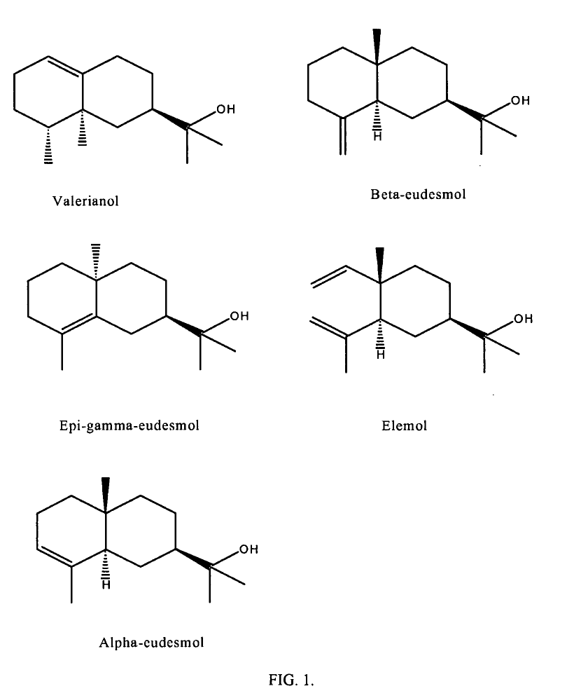

Amyris oil is rich in sesquiterpene alcohols (60-80%), e.g., valerianol, eudesmol (α, β and γ isomers) and elemol (Table 1; Bauer et al., 1990). The structures of these compounds are shown in FIG. 1. The volatile compounds had been identified in the leaf oil of Amyris balsamifera from Cuba (Pino et al., 2006). Fifty-six constituents were identified which constituted more than 95% of the oil composition. The oil was dominated by sesquiterpene alcohols, particularly by valerianol (43.8%), with lesser amounts of γ-eudesmol (15.4%).

TABLE 1
Typical gas chromatography analysis of Amyris Oil
(Alcohol Content from 2 different manufacturers)

| Constituent | Percentage | Percentage |
|---|---|---|
| elemol | 8.99 | 10.0 |
| eudesmol | 42.12 | 27.7 |
| valerianol | 17.04 | 22.1 |
| Selin-5-en-7-ol | | 6.4 |
| Total Alcohol Content | 69.45 | 66.2 |

Green Valley Aromatherapy Ltd., 420 Fitzgerald Avenue, Courtenay, British Columbia, CANADA V9N7N2.

β-eudesmol, is known to have various unique effects on the nervous system. β-eudesmol at concentrations of 100 and 150 μM significantly induced neurite extension in PC-12 cells, which was accompanied, at the highest concentration, by suppression of [³H]thymidine incorporation. Beta-Eudesmol, being a small molecule, may therefore be a promising lead compound for potentiating neuronal function (Yutaro et al., 2002). Proliferation of porcine brain microvascular endothelial cells and human umbilical vein endothelial cells (HUVEC) was inhibited by β-eudesmol (50-100 microM). It also inhibited the HUVEC migration stimulated by basic fibroblast growth factor (bFGF) and the tube formation by HUVEC in Matrigel. β-eudesmol (100 microM) blocked the phosphorylation of extracellular signal-regulated kinase (ERK) ½ induced by bFGF or vascular endothelial growth factor. Furthermore, β-eudesmol significantly inhibited angiogenesis in subcutaneously implanted Matrigel plugs in mice and in adjuvant-induced granuloma in mice. These results indicate that β-eudesmol inhibits angiogenesis, at least in part, through the blockade of the ERK signaling pathway (Tsuneki et al., 2005).

α-eudesmol, which potently inhibits the presynaptic omega-agatoxin IVA-sensitive (P/Q-type) Ca²⁺ channel and neurogenic inflammation following electrical stimulation of rat trigeminal ganglion. It has been suggested that the omega-agatoxin IVA-sensitive Ca²⁺ channel blocker, α-eudesmol, may become useful for the treatment of the neurogenic inflammation in the trigemino-vascular system such as migraine (Asakura et al., 2000). It has been shown that β-Eudesmol blocks the neuromuscular junction. Like phencyclidine, β-eudesmol blocked the nicotinic ACh receptor channel in both the open and closed conformations, and accelerated the desensitization of the nicotinic ACh receptor (Kimura et al., 1991).

α-eudesmol is a Ca²⁺ channel blocker and neurogenic vasodilator, which is useful for treatment of neurogenic inflammation in trigemino-vascular system such as migraine (Asakura et al., 2000a). It attenuates post-ischemic brain injury by reducing the extra cellular glutamate (Asakura et al., 2000b). β-eudesmol is an antidote for intoxication from organophosphorous anti-choline esterase agents (Chiou et al., 1995). It could be used as anti-epileptic (Chiou et al., 1997), neuromuscular blocker (Kimura et al., 1994; 1995) or in treatment of peptic ulcer (Nogami et al., 1986). β-Eudesmol inhibited Na⁺, K(+)-ATPase activity most strongly among the various kinds of phosphatases examined (Satoh et al., 1992).

The chemical formula for eudesmol is C₁₅H₂₆O and that of valerianol is C₁₅H₂₆O and the chemical structures are shown in FIG. 1.

The alcohols present in amyris oil, hereinafter will be called as amyris alcohol, may be obtained by fractional distillation of the oil, with the eudesmol and valerianol appearing in different ratios. The amyris alcohol is colorless to pale yellow in appearance.

Amyris oil is commonly used in the flavor and fragrance industries as a replacement for sandalwood oil and is considered woody, cedar-like, warm and herbaceous. As such, they are essentially non-toxic and harmless when used either for external application on the skin or internal consumption for flavor.

Amyris alcohol is obtained from amyris oil by vacuum distillation by removing the volatile terpenes which are low boiling than the alcohols present in the oil.

Since amyris alcohol contains 80-95% of alcohol, excess application to the skin may cause irritation and itching. To eliminate the irritation and itching, the alcohols can be esterified as they are milder to the skin.

### II. AMYRIS ALCOHOL DERIVATIVES

Various amyris alcohol derivatives, such as esterified amyris alcohols, may be used with the present invention. The ester derivatives of amyris alcohol are capable of reverting to the active parent compound following enzymatic or chemical hydrolysis. These derivatives may have a higher lipophilicity, lipid solubility and be less irritating to the skin than the corresponding amyris alcohol or parent compound. Hence, these ester derivatives may be better suited for incorporation into certain pharmaceutical formulations, such as cream and ointment pharmaceutical formulations. The compounds of the present invention are set forth by the following formulae:

R—CO—O₁Am (I)

wherein O₁Am refers to an oxygen present in an alcohol group of the corresponding unesterified amyris alcohol.

In formula I, R may be an alkyl group, an aryl group, an alkylene group (e.g., an alkenyl or alkynyl), aralalkyl, heteroalkyl, or an arylene group, each of which may vary in size, e.g., C₁-C₂₂, C₁-C₁₈, C₁-C₁₂, C₁-C₆. In certain embodiments, the aryl or aralkyl group is C≦22, C≦18, C≦12, C=6 or C=7. The alkyl, aryl and alkylene groups may be substituted or unsubstituted, branched or straight chains. In addition, R may contain heteroatoms and may be straight chained or branched.

Examples of suitable straight-chain alkyl groups in formula I include methyl, ethyl, propyl, butyl, hexyl, heptyl, octyl, dodecyl, 1-pentadecyl, 1-heptadecyl and the like groups. Examples of suitable branched chain alkyl groups in formula I include isopropyl, sec-butyl, t-butyl, 2-methylbutyl, 2-pentyl, 3-pentyl and the like groups. Examples of suitable cyclic alkyl groups in formula I include cyclopropyl, cyclobutyl, cyclopentyl and cyclohexyl groups.

Examples of suitable “alkenyl” groups in formula I include vinyl(ethenyl), 1-propenyl, i-butenyl, pentenyl, hexenyl, n-decenyl and c-pentenyl and the like.

The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

By the expression “phenalkyl groups wherein the alkyl moiety contains 1 to 3 or more carbon atoms” is meant benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” in formula I are pyridinyl, thienyl or imidazolyl.

As noted herein, the expression “halo” is meant in the conventional sense to include F, Cl, Br, and I.

Among the compounds represented by the general formula I, preferred compounds are such in which R is one of the following groups: methyl, ethyl, propyl, butyl, pentyl, hexyl, 1-pentadecyl, 1-heptadecyl, isobutyl, methoxyethyl, ethoxyethyl, benzyl and nicotinyl.

The compounds of formula I are esters of alcohols present in amyris alcohol. To the knowledge of the inventors, there has been no previous indication in the literature that amyris alcohol, the esters of amyris alcohols, eudesmol, or valerianol have any utility for oral and topical delivery for treating diseases such as cold sores and genital herpes. Without wishing to be bound by any theory, it is anticipated that the esterified amyris alcohols may be enzymatically cleaved once administered to a mammal or human patient in vivo.

A. Methods of Synthesis

The compounds of the present invention can be prepared by any method known to those of ordinary skill in the art. For example, the compounds of the present invention are esters of alcohols which are the constituents of amyris alcohol. Various methods have been described in the literature pertaining to the synthesis of a number of esters of carboxylic acids and alcohols (e.g., March's Advanced Organic Chemistry: Reactions, Mechanisms, and Structure, 5th Edition, by Michael B. Smith and Jerry March, John Wiley and Sons, Inc, 2001, which is incorporated by reference in its entirety). Amyris alcohols and/or esterified amyris alcohols may be purified and used with the present invention.

Various approaches may be used to produce esterified amyris alcohols. Since amyris alcohol is a mixture of tertiary alcohols, esterification can be accomplished using the following procedure. The alcohol can be converted to lithium alcoholate using equimolar amount of either methyl-lithium or t-butyl-lithium under dry and nitrogen atmosphere. The resulting alcoholate can be allowed to react with an equimolar amount of an acyl chloride in diethyl ether under dry condition to produce the desired ester quantitatively. The resulting ester can be vacuum distilled for further purification.

For example, the following protocol may be used in certain embodiments to produce esterified amyris alcohols. A mixture of 100 ml (˜0.4M of alcohol content) of amyris alcohol (Texarome Inc, Leakey, Tex.), 190 ml (2M) of acetic anhydride and 5 drops of H₃PO₄ (85% in water) may be introduced in a 1000 ml flask, and the mixture may be stirred over night, at room temperature. Afterwards, 2 L of water may be added and the stirring may be prolonged for an additional period of 2 hours. The crude product may be extracted by washing the water solution with 1 L of n-hexane. The organic phase thus obtained may be washed twice with a saturated NaHCO₃ water solution, then twice with brine and finally dried over anhydrous MgSO₄ and concentrated. 130 g of crude product (95% yield) having a GC purity of >90% may be obtained using this approach. In various embodiments, propionic anhydride may be used instead of acetic anhydride.

B. Chemical Group Definitions

As used herein, “hydrogen” means —H; “hydroxy” means —OH; “oxo” means ═O; “halo” means independently —F, —Cl, —Br or —I; “amino” means ═NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylamino); “cyano” means —CN; “azido” means —N₃; “mercapto” means —SH; “thio” means ═S; “sulfonamido” means —NHS(O)₂— (see below for definitions of groups containing the term sulfonamido, e.g., alkylsulfonamido); “sulphonyl” means —S(O)₂— (see below for definitions of groups containing the term sulphonyl, e.g., alkylsulphonyl); and “silyl” means —SiH₃ (see below for definitions of group(s) containing the term silyl, e.g., alkylsilyl).

For the groups below, the following parenthetical subscripts further define the groups as follows: “(Cn)” defines the exact number (n) of carbon atoms in the group; “(C≦n)” defines the maximum number (n) of carbon atoms that can be in the group; (Cn-n′) defines both the minimum (n) and maximum number (n′) of carbon atoms in the group. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3-10 carbon atoms)). Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3-10 carbon atoms)).

The term “alkyl” when used without the “substituted” modifier refers to a non-aromatic monovalent group, having a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group, having a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkenyl” when used without the “substituted” modifier refers to a monovalent group, having a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group, having a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkynyl” when used without the “substituted” modifier refers to a monovalent group, having a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CCH₃, are non-limiting examples of alkynyl groups. The term “substituted alkynyl” refers to a monovalent group, having a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “aryl” when used without the “substituted” modifier refers to a monovalent group, having a aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group, having a aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S, Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “aralkyl” when used without the “substituted” modifier refers to the monovalent group—alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group, having a aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group, having a aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

An “isomer” of a first compound is a separate compound in which each molecule contains the same constituent atoms as the first compound, but where the configuration of those atoms in three dimensions differs.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (P. H. Stahl & C. G. Wermuth eds., Verlag Helvetica Chimica Acta, 2002), which is incorporated herein by reference.

“Prevention” or “preventing” when used in reference to a disease includes: (1) inhibiting the onset of the disease in a subject or patient which may be predisposed to the disease but does not yet experience or display the pathology or symptomatology of the disease, (2) slowing the onset of the pathology or symptomatology of the disease in a subject of patient which may be predisposed to the disease but does not yet experience or display the pathology or symptomatology of the disease.

“Prodrug” means a compound that is convertible in vivo metabolically into an inhibitor according to the present invention. For example, prodrugs of amyris alcohols are presented herein, and it is envisioned that a variety of amyris alcohol derivatives or prodrug may be used with the present invention. The prodrug itself may or may not also have activity with respect to a given target protein or therapeutic effect. For example, a compound comprising a hydroxy group may be administered as an ester that is converted by hydrolysis in vivo to the hydroxy compound. As described herein, amyris alcohol prodrugs such as esterified amyris alcohols are provided for the treatment of diseases including herpes virus infection. Suitable esters that may be converted in vivo into hydroxy compounds include acetates, citrates, lactates, phosphates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Amyris alcohols may be esterified using any of these approaches, and it is envisioned that these esterified amyris alcohols may be used with the present invention (e.g., to treat a herpesvirus infection, etc.) Similarly, a compound comprising an amine group may be administered as an amide that is converted by hydrolysis in vivo to the amine compound.

The term “saturated” when referring to a atom means that the atom is connected to other atoms only by means of single bonds.

The terms “subject” and “patient” includes humans, primates and other mammals.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

“Therapeutically effective amount” means that amount which, when administered to an animal for treating a disease, is sufficient to effect such treatment for the disease.

“Treatment” or “treating” includes: (1) inhibiting a disease in an subject or patient that is experiencing or displaying the pathology or symptomatology of the disease (i.e., arresting further development of the pathology and/or symptomatology), and (2) ameliorating the disease in a subject or patient that is experiencing or displaying the pathology or symptomatology of the diseased (i.e., reversing the pathology and/or symptomatology).

### III. PHARMACEUTICAL COMPOSITIONS

Certain embodiments of the present invention pertain to pharmaceutical compositions comprising the esters of amyris alcohols set forth herein. A variety of drug delivery systems may be used with the present invention, including topical and transdermal drug delivery systems.

The phrases “pharmaceutical,” “pharmaceutically,” or “pharmacologically acceptable” refer to molecular entities and compositions that do not produce an unacceptably adverse, allergic or other untoward reaction when administered to an animal, or human, as appropriate. As used herein, “pharmaceutical” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients to treat the disease of interest, such as other anti-cancer agents or anti-inflammatory agents, can also be incorporated into the compositions.

The preparation of an pharmaceutical composition that contains at least one amyris alcohol or esterified amyris alcohol or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

Pharmaceutical compositions of the present invention will include an effective amount of one or more of the ester derivatives set forth herein that is clinically determined to be useful in the treatment of the particular disease under consideration. One of ordinary skill in the art would be familiar with determination of appropriate dosages which could be used for treatment of the particular pathological condition that is present in the subject. In the case of topical application, for example, the amount of amyris alcohol or esterified amyris alcohol present in the formulation can vary, for example, from about 1% to about 25% by weight, from about 5% to about 20% by weight, or from about 10% to about 15% by weight. When used for therapy, the compositions of the present invention are administered to subjects in therapeutically effective amounts. For example, an effective amount of the ester of amyris alcohol in a patient with cold sores may be an amount that promotes the healing of the sores. The dose will depend on the nature of the disease, the subject, the subject's history, and other factors. Preparation of such compositions is discussed in other parts of this specification.

As discussed above, the derivatives set forth herein have greater lipophilicity and less irritation to the skin than amyris alcohols. One advantage of these esters is that they can be incorporated into a cream or ointment form at a higher percentage by weight as compared to amyris alcohols. Another advantage is that these compositions have a very low toxicity and irritation to the skin as compared to formulations of amyris alcohol.

It is anticipated that the amyris alcohol derivatives of the present invention may be delivered by virtually any method known to those of ordinary skill in the art. For example, the pharmaceutical compositions can be delivered by topical or oral delivery routes.

Compositions employing the esters of amyris alcohol set forth herein will contain a biologically effective amount of the derivative. As used herein a “biologically effective amount” of a compound or composition refers to an amount effective to alter, modulate or reduce disease conditions. One of ordinary skill in the art would be familiar with methods of determining a biologically effective amount of a therapeutic agent. For example, a biologically effective amount may be about 0.1 mg/kg to about 50 mg/kg or greater

A therapeutic ester of an amyris alcohol of the present invention may be administered alone or in combination with one or more additional therapeutic esters of the present invention. In other embodiments, a therapeutic ester of amyris alcohol is administered in combination with one or more secondary forms of therapy directed to the disease or condition to be treated. These are discussed in greater detail below. Additional pharmaceutical compounds may be administered in the same pharmaceutical composition, or in a separate dosage form, such as in a separate oral, intramuscular, or intravenous dosage forms taken at the same time.

The therapeutic agents of the present invention may be supplied in any form known to those of ordinary skill in the art. For example, the therapeutic agent may be supplied as a liquid or as a solution. The pharmaceutical compositions may contain a preservative to prevent the growth of microorganisms. It must be chemically and physically stable under the conditions of manufacture and storage and must be preserved against the contaminating action of microorganisms, such as bacteria and fungi. The prevention of the action of microorganisms can be brought about by various antibacterial and antifungal agents, for example, parabens, chlorobutanol, phenol, sorbic acid, thimerosal, and the like.

The formulations according to the invention having been described herein may influence the ordinarily skilled artesian to make similar formulations using components that will be known in the art, without departing from the invention which is claimed herein.

The pharmaceutical formulations of the esters of amyris alcohol according to the present invention offer several advantages over the existing formulations. They can be topically applied and relatively high concentrations of the esters of amyris alcohol can be loaded into patients with high bioavailability. For example, a topical gel containing from about 10% to 20% by weight of either amyris alcohol or an ester of amyris alcohol can be used. Thus the frequency of dosage can be reduced. As mentioned herein above a number of excipients may be included in formulations of the present invention. The inclusion of excipients and the optimization of their concentration for their characteristics such as for example ease of handling or carrier agents will be understood.

Following preparation of the pharmaceutical compositions of the present invention, it may be desirable to quantify the amount of the esters of amyris alcohol in the pharmaceutical composition. Methods of measuring concentration of a drug in a composition include numerous techniques that are well-known to those of skill in the art, including chromatographic techniques such as: drug-specific assays, adsorption, partition, ion-exchange and molecular sieve, and many specialized techniques for using them including column, paper, thin-layer chromatography, gas chromatography, and high performance liquid chromatography (HPLC). One of ordinary skill in the art would be familiar with these and other related techniques.

A. Moisturizing Agents

Certain topical formulations of the present invention may contain moisturizing agents. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, β-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copemicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, fructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquarternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

B. Antioxidants

Certain topical formulations of the present invention may also contain one or more antioxidants. Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

### IV. PATHOLOGICAL CONDITIONS TO BE TREATED OR PREVENTED

As noted in other parts of this specification, there is substantial evidence that amyris alcohol would be beneficial in the treatment of a wide variety of pathological conditions. The term “treat” or “treatment” means that the symptoms associated with one or more conditions mentioned above are alleviated or reduced in severity or frequency and the term “prevent” means that subsequent occurrences of such symptoms are avoided or that the frequency between such occurrences is prolonged.

Examples of pathological conditions responsive to amyris alcohol therapy include, but are not limited to, gonorrhea, bronchitis, sore throat, persistent cough, fever, pain, herpes infection caused by HSV-1 and HSV-2 virus and warts caused by the human papillomavirus (HPV) in humans (Kaur 2005). For example, in test tube studies, sandalwood oil, which contains santalol, was found to slow the growth of herpes virus (Benencia 1999). An intriguing animal study found that components isolated from sandalwood caused responses similar to those seen with medications used to treat schizophrenia (Okugawa 1995). The sandalwood oil displayed chemoprotective effects on 7,12-dimethylbenz(a)anthracene-(DMBA)-initiated and 12-O-tetradecanoyl phorbol-13-acetate (TPA)-promoted skin papillomas, and TPA-induced ornithine decarboxylase (ODC) activity in mice. Treatment with either sandalwood oil or santalol significantly decreased papilloma incidence by 67%, multiplicity by 96%, and TPA-induced ODC activity by 70% (Dwivedi and Abu-Ghazaleh. 1997; Dwivedi 2003). The sandalwood oil was found to enhance glutathione S-transferase (GST) activity and acid soluble sulphydryl (SH) levels in the liver of adult male Swiss albino mice, suggesting a possible chemopreventive action (Banerjee et al. 1993). Sandalwood oil is said to act as an antiseptic in the urinary system (Blumenthal 1998) and it might help to rid the body of the bacteria that cause these infections (Leung 1996)

### V. COMBINATION THERAPIES

Some embodiments of the claimed methods of the present invention involve administering to the subject a secondary form of therapy in addition to one or more of the therapeutic ester derivatives of amyris alcohol set forth herein. For example, if the disease is a hyperproliferative disease, such as cancer, the secondary therapy may be a chemotherapeutic agent, radiation therapy, surgical therapy, immunotherapy, gene therapy, or other form of anticancer therapy well-known to those of ordinary skill in the art. If the disease is an inflammatory disease such as arthritis, exemplary secondary forms of therapy include non-steroidal anti-inflammatory agents, steroids, and immunosuppressant therapy.

In order to increase the effectiveness of the therapeutic agent disclosed herein, it may be desirable to combine the therapeutic agent of the present invention with the secondary therapeutic agent. These compositions would be provided in a combined amount effective to provide for a therapeutic response in a subject. One of ordinary skill in the art would be able to determine whether the subject demonstrated a therapeutic response. This process may involve administering the therapeutic agent of the present invention and the secondary therapeutic agent to the subject at the same time or sequentially, e.g., within a period of from about 1 minute to about 12 hours. This may be achieved by administering a single composition or pharmacological formulation that includes both agents, or by administering two distinct compositions or formulations, at the same time, wherein one composition includes the curcumin derivative of the present invention and the other includes the secondary agent.

Alternatively, the therapeutic agent of the present invention may precede or follow the treatment with the secondary agent by intervals ranging from about 1 minute to about 2 weeks. In embodiments where the secondary agent and the curcumin derivative of the present invention are separately administered, one would generally ensure that a significant period of time did not expire between the time of each delivery, such that the secondary agent and the therapeutic agent of the present invention would still be able to exert a beneficial effect on the subject. In such instances, it is contemplated that one may administer both modalities within about 24-48 h of each other and, more preferably, within about 12-24 h of each other, and even more preferably within about 30 minute-6 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, the therapeutic agent of the present invention is “A” and the secondary agent, such as chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the present invention to a patient will follow general protocols for the administration of therapeutic agents, such as chemotherapy where the disease to be treated is cancer. It is expected that the treatment cycles would be repeated as necessary.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Acetyl Ester of Amyris Alcohol

### Formula Ia, R═CH₃

A mixture of 100 ml (˜0.4M of alcohol content) of amyris alcohol (Texarome Inc, Leakey, Tex.), 190 ml (2M) of acetic anhydride and 5 drops of H₃PO₄ (85% in water) were introduced in a 1000 ml flask, and the mixture was stirred over night, at room temperature. Afterwards, 2 L of water where added and the stirring was prolonged for an additional period of 2 hours. The crude product was extracted by washing the water solution with 1 L of n-hexane. The organic phase thus obtained were washed twice with a saturated NaHCO₃ water solution, then twice with brine and finally dried over anhydrous MgSO₄ and concentrated. It was thus obtained 130 g of crude product (95% yield) having a GC purity of >90%.

A dry, 500-ml., three-necked flask was placed in a water bath which was placed on a magnetic stirrer and was fitted with two pressure-equalizing dropping funnel and an inlet tube to maintain a static nitrogen atmosphere in the reaction vessel throughout the reaction. In the flask were placed 100 gm (˜75% of alcohol content; 0.34M) of amyris alcohol (Texarome Inc, Leakey, Tex.). 113 mL (3M in diethoxymethane) of methyl-lithium solution was transferred to one of the dropping funnel and 24 mL (0.35M) of acetyl chloride was transferred to the other dropping funnel. While the amyris alcohol was stirred, the methyl-lithium solution was added dropwise over a 20-minute period. The resulting mixture was stirred for another 30 minutes, at which time the formation of lithium amyris alcoholate was complete. After the resulting suspension had been cooled to approximately 15° with ice, it was stirred vigorously while acetyl chloride was added dropwise over a 30-minute period. After the addition was complete, the ice bath was removed and the resulting suspension was stirred at room temperature for 2 hours. The dropping funnels were removed and the fine suspension in the reaction flask was agitated and siphoned into a vigorously stirred solution of 400 ml. of 10% sodium carbonate in water. The reaction flask was rinsed with an additional 30 ml. of ether which was also added to the aqueous solution. After the resulting mixture had been saturated with sodium chloride, the organic phase was separated and the alkaline aqueous phase was extracted with three 50-ml. portions of ether. When the combined organic solutions had been dried over magnesium sulfate, the bulk of the ether was distilled from the mixture through a 40-cm. Vigreux column and then the residual ether were distilled from the mixture through a 10-cm. Vigreux column. The residual yellow liquid were distilled under vacuum and the fraction distilled between 100° and 115° was collected as a clean pale yellow oil (yield 50%).

### Example 2

### Preparation of Propanoyl Ester of Amyris Alcohol

### Formula Ia, R═CH₃—(CH₂)

The compound was prepared essentially as described in Example 1, using propionic anhydride instead of acetic anhydride. The product was recovered as a pale yellow oil.

### Example 3

### Preparation of Topical Gel Containing Acetyl Ester of Amyris Alcohol

The following procedure was used to prepare a 10% gel containing acetyl ester of amyris alcohol.

### Preparation of Base Gel, Part A

In a clean container, add and mix

| | | |
|---|---|---|
| Purified water | 600 | ml |
| Potassium sorbate | 1.0 | g |
| Methyl paraben sodium | 2.0 | g |
| Disodium edetate | 5.0 | g |
| Xantahan gum | 3.0 | g |
| Carbopol Ultrez10 | 30.0 | g |

### Preparation of Organic Phase, Part B

In another glass container, add and dissolve at 45-50 degrees C.,

| | | |
|---|---|---|
| Tween 80 | 0.6 | g |
| Hallbrite BHB | 20.0 | g |
| Acetyl ester of amyris alcohol | 100.0 | g |
| Eugenyl Acetate | 10.0 | g |
| Ascorbyl Palmitate | 5.0 | g |
| Isobornyl acetate | 5.0 | g |
| Cetyl palmitate | 10.0 | g |

(The above method can be modified by substituting 100.0 g of a propanoyl ester of amyris alcohol instead of the acetyl ester of amyris alcohol.)

Add part B to Part A and mix well in a blender

Add and mix,

| | | |
|---|---|---|
| Triethanolamine | 16.0 | g |
| Purified water to q.s | 1000 | g |

Further homogenize the gel in a high pressure homogenizer.
The pH of the gel is 6.0-6.5. The gel is smooth and white in color.

### Example 4

### Preparation of Topical Gel Containing Amyris Alcohol

The following procedure was used to prepare a 15% gel containing propanoyl ester of amyris alcohol.

### Preparation of Base Gel, Part A

In a clean container, add and mix

| | | |
|---|---|---|
| Purified water | 600 | ml |
| Potassium sorbate | 1.0 | g |
| Methyl paraben sodium | 2.0 | g |
| Disodium edetate | 5.0 | g |
| Xantahan gum | 3.0 | g |
| Carbopol Ultrez10 | 30.0 | g |

### Preparation of Organic Phase, Part B

In another glass container, add and dissolve at 45-50 degrees C.,

| | | |
|---|---|---|
| Tween 80 | 0.6 | g |
| Hallbrite BHB | 20.0 | g |
| Amyris alcohol | 150.0 | g |
| Eugenyl Acetate | 10.0 | g |
| Ascorbyl Palmitate | 5.0 | g |
| Isobornyl acetate | 5.0 | g |
| Cetyl palmitate | 10.0 | g |

Add part B to Part A and mix well in a blender
Add and mix,

| | | |
|---|---|---|
| Triethanolamine | 16.0 | g |
| Purified water to q.s | 1000 | g |

Further homogenize the gel in a high pressure homogenizer.
The pH of the gel is 6.0-6.5. The gel is smooth and white in color.

### Example 5

### Preparation of Topical Cream Containing 20% Amyris Alcohol

A skin cream composition containing amyris alcohol is shown in Table 2, and lists the ingredients in the compositions containing amyris alcohol. The top portion of Table 2 shows the proportions of the base, and the bottom portion shows the constituents and proportions of the additives and all proportions are in units of percent by weight. As shown in Table 2, the base consists of a commercially available moisturizing skin lotion and the additive consists of amyris alcohol. The base and the additives were mixed thoroughly in a blender to prepare the cream.

TABLE 2
Topical Cream Composition for of propionyl amyris alcohol

| COMPOSITION | |
|---|---|
| INGREDIENT NAME | % |
| A. BASE INGREDIENT | |
| Lubriderm Moisturizing Lotion | 80.0 |
| sold by Pfizer Healthcare Product | |
| Newhaven, Connecticut | |
| B. ADDITIVES | |
| Amyris alcohol | 20.0 |
| TOTAL ADDITIVES | 20.0 |

### Example 6

### Toxicity Assessment

A 15% topical gel of amyris alcohol as described in example 4, was applied to the forearm of 10 healthy individuals twice daily for a two-week period in an outpatient clinic. No patients complained of burning, irritation, scaling or redness after the cream. Patients returned to the clinic after having used the solution for two weeks for a visual inspection of the forearm area. The examining physician noted no redness, irritation or scaling in the area where the solution had been applied. Compositions comprising amyris alcohols were used in the below examples.

### Example 7

### Case I. Patient with Cold Sores Treated with 15% of Amyris Alcohol

A 22 year old male has developed cold sores on his mouth with reddish blisters and pain. He was given the 15% topical gel of amyris alcohol and the following is his testimony on the effectiveness of the gel for the treatment of cold sores. “I was given a sample gel to treat cold sores I had acquired on my lower lip. I was informed to apply it a couple times a day and it would begin to clear. I began about 7 p.m. by rubbing a small amount of the gel on the surface of the sore. I applied it once more before I slept and again in the morning. By morning time I had noticed a reduction in the color of the sore. It had gone from an unsightly, irritated flesh color to a more neutral tone. I repeated applications twice more that day and by that evening I had noticed a few other details. The product moistened my lip so the scab of the cold sore was not apparent. Tenderness and swelling had diminished as well as the overall diameter of the original sore. Within 24 hours it had appeared that the cold sore was almost gone. My cold sores generally take close to two weeks to heal if left untreated and with this medication it appeared to have disappeared in about 2 days of treatment. By the 48^{th} hour of treatment my sore had been almost completely wiped out and my normal skin tone had returned”.

### Case II. Patient with Cold Sores Treated with 15% of Amyris Alcohol

A 46 year old male developed cold sores on his mouth and was treated with the 15% topical gel of amyris alcohol. He gave the following testimony about the treatment. “I recently tried a topical solution for the treatment of cold sores/fever blisters on my lip. I applied a small amount of the gel (approximately 0.5 gm) to the cold sores/fever blisters then rubbed the solution into them and immediately experienced a drying effect of theses cold sores/fever blisters. I repeated this application a few times a day and within three to four days the cold sores/fever blisters were healed. I experience theses cold sores/fever blisters every couple of years or so, I do not suffer from them on a regular basis. They occur when I become run down from a combination of over working, lack of sleep, poor diet and lack of exercise; basically when I run my immune system down. I have tried over the counter medication and prescription medications in the past and nothing has worked so effectively as the solution I received. I would recommend this solution to anyone who suffers from occasional or reoccurring cold sores/fever blisters. These sores are embarrassing and debilitating to a person's self-confidence and their ego. The gel is extremely effective and works quickly to dry and clear the sores. If any additional information is required, I will make myself available to inquiry.

### Case III. Patient with Cold Sores Treated with 15% of Amyris Alcohol

A 41 year old female developed cold sores on her mouth and was treated with the 15% topical gel of amyris alcohol. She gave the following testimony about the treatment. “I use to get cold sores every 3-4 months and it would give me pain and redness around my mouth. I recently tried a topical cream for the treatment of cold sores/fever blisters on my lip. I started applying a small amount of the cream to the cold sores/fever blisters then rubbed the cream into them and sensed a drying effect within an hour of theses cold sores/fever blisters. I used this application a few times a day and within three to four days the cold sores/fever blisters were healed. This cream is much better than the medication I have tried before obtained over the counter and nothing has worked so effectively as the cream I used”.

### Case IV. Patient with Herpes Treated with 15% of Amyris Alcohol

A 66 year old female developed herpes outbreak around her vaginal area and was treated with the 15% topical gel of amyris alcohol. She gave the following testimony about the treatment. “I used to get herpes outbreak around my vaginal area with blisters few times every year and I used the cream provided to me to treat my herpes blisters. I applied the thoroughly on the affected area 2-3 times a day and I noticed that the blisters started drying within 3 days. After a week of application, it is completely gone and I would be very happy to recommend this wonderful cream for any person who suffers from herpes blisters”.

### Case V. Patient with Cold Sores Herpes Treated with 15% Cream of Amyris Alcohol

A female clinical surgical nurse had cold sore blisters and she was provided with the 15% cream of amyris alcohol. She had provided the following testimony. “I have struggled with cold sores on my lower lip since I was 18 years old. I have tried every over the counter product out there and prescription medicines. In the past, it usually takes anywhere from one to two weeks for the cold sore to heal stop hurting. Many of the medicines, I have tried have caused quite a bit of discomfort upon application. I had convinced myself that there was not anything out there that could painlessly heal my cold sore in less than a week. I was overjoyed when I tried the cold sore cream given to me as a sample. This cream did not cause pain upon application and kept my lip soft through out the healing process. Amazingly, my cold sore was completely gone in about 30-36 hours. I have never had cold core heal that quickly. This cold sore cream also has a very pleasant taste. Thank you for allowing me to try this cream.”

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Asakura et al., Brain Res., 873:94-101, 2000.
Bauer et al., In: Common fragrance and flavor materials: preparation, properties and uses, 2^{nd} Ed., Weinheim, VCH, 1990.
Benencia and Courrèges, Phytomedicine, 6(2):119-123, 1999.
Boon et al., Clinical Therapeutics, 22(1):76-90, 2000.
Chiou et al., Eur. J. Pharmacol., 13:151-156, 1995.
Chiou et al., Neurosci. Lett., 15:171-174, 1997.
FDA Office of Biological Standards
Habif et al., In: Skin Disease Diagnosis and Treatment, 160-163, Mosby (Ed.), St. Louis, 2001.
Habif, In: Herpes simplex. In Clinical Dermatology: A Color Guide to Diagnosis and Therapy, 4^{th} Ed., 381-387, Mosby (Ed.), Philadelphia, 2004.
Handbook of Pharmaceutical Salts: Properties, Selection and Use, Stahl and Wermuth (Eds.) Verlag Helvetica Chimica Acta, 2002.
Herpes labialis, Clinical Evidence, (9):1836-1841, 2003.
Kimura et al., Neuropharmacology, 30:835-841, 1991.
March's Advanced Organic Chemistry: Reactions, Mechanisms, and Structure, 5^{th} Ed., Smith and March (Eds.), John Wiley and Sons, Inc, 2001.
Nogami et al., Chem. Pharm. Bull., 34:3854-3860, 1986.
Pino et al., In: Aromatic Plants from Western Cuba. VI. Composition of the Leaf Oils of Murraya exotica L., Amyris balsamifera L., Severinia buxifolia (Poir.) Ten. and Triphasia trifolia (Burm. f.), J. Essential Oil Research: JEOR, 2006.
Remington's Pharmaceutical Sciences, 18th Ed. Mack Printing Company, 1990,
Sacks et al., J. Amer. Acad. Derma., 45:222-230, 2001.
Satoh et al., Biochem. Pharmacol., 44:373-378, 1992.
Van et al., J. Essent. Oil Res., 3:127-128, 1991b.
Van et al., Phytochemistry, 28(7):1909-1912, 1989.
Yutaro et al., Pharmacol. Experim. Therap., 301:803-811, 2002.
Kimura et al., Biol. Pharm. Bull., 17:1232-1240, 1994.
Kimura et al., Biol. Pharm. Bull., 18(5):691-695, 1995.
Dwivedi and Abu-Ghazaleh, Eur. J. Cancer Prev., 6(4):399-401, 1997.
Tsuneki et al., Eur. J. Pharmacol., 512(2-3):105-115, 2005.
Banerjee et al., Cancer Lett., 68(2-3):105-109, 1993.
Van et al., J. Essent. Oil Res., 3:59-60, 1991a.
Asakura et al., Eur. J. Pharmacol., 7:57-65, 2000b.
Okugawa et al., Phytomedicine. 2:119-126, 1995.
Blumenthal Ed. The Complete German Commission E Monographs, Therapeutic Guide to Herbal Medicines. Austin, Tex.: American Botanical Council; Boston, Mass.: Integrative Medicine Communications; 1998: 118.
Leung et al., Encyclopedia of Common Natural Ingredients Used in Food, Drugs, and Cosmetics. New York, N.Y.: John Wiley & Sons; 1996: 460-461.

1. A method for treating a disease comprising administering to a mammal an amyris alcohol or an esterified amyris alcohol of formula (I):
R—CO—O₁Am (I)
wherein O₁Am refers to an oxygen present in an alcohol group of the corresponding non-esterified amyris alcohol, wherein R is selected from the group consisting of C₁-C₁₈ alkyl, C₁-C₁₈ aryl, C₁-C₁₈ alkylene, C₁-C₁₈ substituted alkyl, C₁-C₁₈ substituted aryl, and C₁-C₁₈ substituted alkylene; wherein if an amyris alcohol is administered, then the mammal is a human and the disease is selected from the group consisting of cold sores, genital herpes, herpes simplex virus infection, HSV-1 infection, HSV-2 infection, epidermoid carcinoma, and human papillomavirus (HPV) tumors.

2. The method of claim 1, wherein the amyris alcohol is administered to the human.

3. The method of claim 2, wherein the amyris alcohol is selected from the group consisting of valerianol, beta-eudesmol, epi-gamma-eudesmol, elemol, or alpha-eudesmol.

4. The method of claim 1, wherein the esterified amyris alcohol is administered to the mammal.

5. The method of claim 4, wherein the disease is selected from the group consisting of cold sores, genital herpes, herpes simplex virus infection, HSV-1 infection, HSV-2 infection, epidermoid carcinoma, and human papillomavirus (HPV) tumors.

6. The method of claim 4, wherein R is selected from methyl, ethyl, propyl, butyl, hexyl, heptyl, octyl, dodecyl, 1-pentadecyl, 1-heptadecyl, isopropyl, sec-butyl, t-butyl, 2-methylbutyl, 2-pentyl, 3-pentyl, cyclopropyl, cyclobutyl, cyclopentyl and cyclohexyl, vinyl(ethenyl), 1-propenyl, i-butenyl, pentenyl, hexenyl, n-decenyl and c-pentenyl groups.

7. The method of claim 1, wherein the esterified amyris alcohol is administered to the mammal, and wherein the esterified amyris alcohol is selected from the group consisting of:

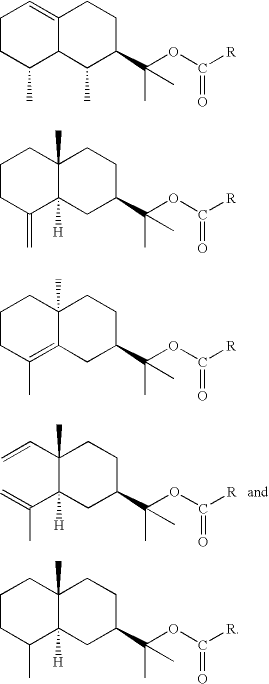

8. The method of claim 7, wherein R is selected from methyl, ethyl, propyl, butyl, hexyl, heptyl, octyl, dodecyl, 1-pentadecyl, 1-heptadecyl, isopropyl, sec-butyl, t-butyl, 2-methylbutyl, 2-pentyl, 3-pentyl, cyclopropyl, cyclobutyl, cyclopentyl and cyclohexyl, vinyl(ethenyl), 1-propenyl, i-butenyl, pentenyl, hexenyl, n-decenyl and c-pentenyl groups.

9. The method of claim 1, wherein said disease is a tumor induced by a human papillomavirus (HPV) selected from the group consisting of verrucae warts, plantar warts, flat warts, genital warts and Molluscum contagiosum.

10. The method of claim 1, wherein the pharmaceutical composition is comprised in a topical formulation.

11. The method of claim 10, wherein the topical formulation is a cream, lotion, spray, wipe, or drop formulation.

12. The method of claim 1, wherein the pharmaceutical composition comprises one or more additional pharmaceutical agents.

13. The method of claim 12, wherein the one or more additional pharmaceutical agents includes a fungicidal or fungistatic agent, a bacteriocidal or bacteriostatic agent, a viricidal or virostatic agent, or a cytotoxic agent.

14. The method of claim 1, wherein the composition is a pharmaceutical composition further comprising one or more pharmaceutically acceptable excipients.

15. The method of claim 14, wherein the excipients include one or more pharmaceutically acceptable antioxidants.

16. The method of claim 15, wherein the antioxidant is ascorbic acid, sodium ascorbate, sodium bisulfite, sodium metabisulfate, curcumin, curcumin derivatives, ursolic acid, resveratrol, resveratrol derivatives, alpha-lipoic acid or monothioglycerol.

17. The method of claim 14, wherein the excipients include one or more pharmaceutically acceptable preservatives and/or buffering agents.

18. The method of claim 17, wherein the buffering agent is monobasic and dibasic sodium phosphate, sodium benzoate, potassium benzoate, sodium citrate, sodium acetate or sodium tartrate.

19. The method of claim 17, wherein the preservative is methylparaben, methylparaben sodium, propylparaben, propylparaben sodium, benzalkonium chloride or benzthonium chloride.

20. The method of claim 1, wherein the composition comprises one or more pharmaceutically acceptable polysaccharides.

21. The method of claim 20, wherein the polysaccharide is dextran sulfate, pectin, modified pectin, insoluble 1,3-β-D glucan, micronized 1,3-β-D glucan, soluble 1,3-β-D glucan, phosphorylated 1,3-β-D glucan, aminated 1,3-β-D glucan and carboxymethylated 1,3-β-D glucan, sulfated 1,3-β-D glucan, insoluble 1,3/1,6-β-D glucan, micronized 1,3/1,6-β-D glucan, soluble 1,3/1,6-β-D glucan, phosphorylated 1,3/1,6-β-D glucan, aminated 1,3/1,6-β-D glucan and carboxymethylated 1,3/1,6-β-D glucan or sulfated 1,3/1,6-β-D glucan.

22. The method of claim 1 wherein the mammal is a human.

23. The method of claim 1 wherein said amyris alcohol is obtained from Amyris balsamifera.

24. The method of claim 1, wherein the pharmaceutical composition comprises from about 1% to about 90% by weight of the amyris alcohol or an ester of amyris alcohol.

25. The method of claim 24, wherein the pharmaceutical composition comprises from about 5% to about 50% by weight of amyris alcohol or an ester of amyris alcohol.

26. The method of claim 25, wherein the pharmaceutical composition comprises from about 10% to about 30% by weight of amyris alcohol or an ester of amyris alcohol.

27. The method of claim 1, wherein the composition is administered orally, nasally, topically, rectally or vaginally.

28. A composition comprising an esterified amyris alcohol of formula (I):
R−CO—O1Am (I)
wherein O1 refers to an oxygen present in an alcohol group of the corresponding non-esterified amyris alcohol, wherein R is selected from the group consisting of C1-C18 alkyl, C1-C18 aryl, C1-C18 alkylene, C1-C18 substituted alkyl, C1-C18 substituted aryl, and C1-C18 substituted alkylene.

29. The composition of claim 28, wherein R is selected from methyl, ethyl, propyl, butyl, hexyl, heptyl, octyl, dodecyl, 1-pentadecyl, 1-heptadecyl, isopropyl, sec-butyl, t-butyl, 2-methylbutyl, 2-pentyl, 3-pentyl, cyclopropyl, cyclobutyl, cyclopentyl and cyclohexyl, vinyl(ethenyl), 1-propenyl, i-butenyl, pentenyl, hexenyl, n-decenyl and c-pentenyl groups.

30. The composition of claim 28, wherein the esterified amyris alcohol is selected from the group consisting of:

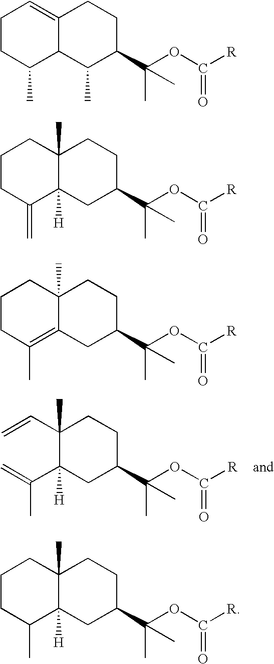

31. The composition of claims 30, wherein R is selected from methyl, ethyl, propyl, butyl, hexyl, heptyl, octyl, dodecyl, 1-pentadecyl, 1-heptadecyl, isopropyl, sec-butyl, t-butyl, 2-methylbutyl, 2-pentyl, 3-pentyl, cyclopropyl, cyclobutyl, cyclopentyl and cyclohexyl, vinyl(ethenyl), 1-propenyl, i-butenyl, pentenyl, hexenyl, n-decenyl and c-pentenyl groups.

32. The composition of claim 28, wherein the composition is a pharmaceutical preparation.

33. The composition of claim 32, wherein the pharmaceutical preparation is sterile or a Good Manufacturing Practice (GMP grade) pharmaceutical preparation.

34. The composition of claim 28, wherein the composition is a cosmetic or topical composition.

35. The composition of claim 33, wherein the composition is a topical composition selected from the group consisting of an emulsion, a cream, a lotion, a solution, an anhydrous composition, a gel, or an ointment.

36. The composition of claim 35, wherein the composition is a topical gel.
